(19) United States
(12) Patent Application Publication
(10) Pub. No.: US 2008/0195326 A1
(43) Pub. Date: Aug. 14, 2008

(54) Method And System For Comprehensive Knowledge-Based Anonymous Testing And Reporting, And Providing Selective Access To Test Results And Report

(76) Inventors: Martin Munzer, Coral Springs, FL (US); Keith Zucker, Fresno, CA (US); Eduard Peter-Shofen, Oldenburg (DE); Andrea Doescher, Oldenburg (DE)

Correspondence Address: ARENT FOX LLP, 1050 CONNECTICUT AVENUE, N.W., SUITE 400, WASHINGTON, DC, 20036, US

(21) Appl. No.: 11/579,485

(22) PCT Filed: May 3, 2005
(86) PCT No.: PCT/US05/15328

§ 371 Date: Jan. 25, 2008

Related U.S. Application Data
(60) Provisional application No. 60/566,922, filed on May 3, 2004.

(51) Int. Cl. G06F 17/00; C12Q 1/68; C12M 1/00; G06Q 30/00

(52) U.S. Cl. 702/20; 435/6; 435/287.2; 705/34

(57) ABSTRACT

A method and system (100) for comprehensive knowledge-based genetic and other testing and for providing selective access to testing results (120) thereof. In particular, a method and system is provided for the personal, anonymous and confidential examination of the genetic profile of any gene or combination of genes and examining any mutations of such genes to assess the risk of developing a condition, such as a medical disease, or to identify personal characteristics and/or traits. Other data, such as chemical analysis, environmental, personal and family history of medical, behavioral, and other factors can be incorporated to generate a comprehensive knowledge-based assessment. The method and system also include features for providing secure confidential access to such assessments to third parties (160).

### CROSS-REFERENCE TO RELATED APPLICATIONS

The present invention claims priority to and is related to U.S. provisional application No. 60/566,922, filed May 3, 2004, the entirety of which is hereby incorporated by reference in its entirety.

### BACKGROUND OF THE INVENTION

1. Field of the Invention

The present invention relates to the method and system for comprehensive knowledge-based testing and providing selective access to testing results. In particular, the present invention provides a method and system for the personal, anonymous and confidential examination of the genetic profile of a gene or combination of genes and examining the mutation(s) of the gene(s) to assess the risk of developing a condition, such as a medical disease, or to identify personal characteristics and/or traits. Other data, such as laboratory chemical analysis of blood, urine and other biological materials, environmental, personal and family history of medical, behavioral, and other factors can be incorporated in the method and system to generate a comprehensive knowledge-based assessment. Furthermore, the present invention provides secure confidential access to such assessment.

2. Description of the Related Art

Information regarding personal health and history can be very important and helpful in maintaining health and preventing illnesses and disease. More and more individuals are seeking better ways to care for themselves by being informed, getting regular physical examinations, and having periodic medical testing and check-ups.

Genetic profiling is a process of analyzing an individual's genetic material, which can have a variety of applications, many of which are medical in nature. For example, genetic profiling can be used to determine whether an individual has a genetic predisposition to a particular disease. Currently, genetic profiling is performed mainly in research laboratories for research purposes.

In recent years, consumer confidence in the healthcare system has eroded. Rather than relying solely on the healthcare system, individuals are seeking tools to empower themselves to manage their own healthcare. Therefore, there is a need to provide a confidential and personal genetic profiling system and method that allows individuals to obtain a genetic profile for themselves, and access such results to better understand their health.

### SUMMARY OF THE INVENTION

The present invention provides a method and system for generating a comprehensive knowledge-based report of an individual using results from a genetic profile test, together with other relevant factors, and providing the results of the test to the individual. The present method and system are particularly useful when used to detect a condition for which the individual can take preventative action. For such purposes,, the system provides the individual with information that can be used to prevent, delay, or decrease the chances of developing a particular condition. The information provided to the individual includes, for example, information concerning diet, exercise, or preventative supplements and drugs. For example, an individual with a predisposition to Alzheimer's disease may increase folic acid intake and physical activity or reduce fat intake to delay the progression of symptoms. Similarly, an individual who is predisposed to osteoporosis because of a mutation in the vitamin D receptor may take vitamin D supplements and perform weight-bearing exercises to help build bone mass. Other preventative actions for other predispositions or conditions similarly determinable.

Other applications of genetic profiling include determining drugs that are well-suited to a particular individual, which may be achieved by conducting a pharmacogenomic profile. Knowing how an individual absorbs, metabolizes, distributes, reacts to, or eliminates certain drugs aids in prescribing medications, and can reduce the risk of overdose, adverse reaction, or interaction with other drugs. For example, individuals who are slow metabolizers of a certain drug, when prescribed at the standard dosage, experience a build-up of the drug in their system and experience toxic side-effects. Slow metabolizers of a particular drug may therefore be prescribed a lower dosage and may therefore avoid toxic side-effects and effect a positive therapeutic response.

Yet another application includes conducting tests on fetal deoxyribonucleic acid (DNA). Fetal DNA can be identified within a blood or urine specimen from the mother, for example. The fetal DNA can then be used to perform genetic profiling on the fetus, and to allow other actions, such as to diagnose and treat medical conditions in the womb. For example, a fetus with a blood type that is incompatible with the mother's blood type is at risk of miscarriage. Genetic profiling can be used to identify the fetal blood type. If the blood type of the fetus is found to be incompatible with that of the mother, corrective measures can be taken to avoid complications. Genetic profiling of the fetus may not only allow the patient to address possible or future health concerns, but also may identify positive attributes, such as the predisposition to becoming a good artist, musician, mathematician or athlete.

### BRIEF DESCRIPTION OF THE DRAWINGS

The foregoing and other objects, features, and advantages of the invention will become more readily apparent upon reference to the following detailed description of a presently preferred embodiment, when taken in conjunction with the accompanying drawings in which:

FIG. 1 illustrates a flow diagram showing one example the genetic profile testing method in accordance with one embodiment of the present invention;

FIG. 2 illustrates one example of the system for genetic profile testing and comprehensive knowledge-based analysis in accordance with an embodiment of the present invention;

FIG. 3 illustrates flow diagram showing another example of providing genetic profile testing in accordance with an embodiment of the present invention;

FIG. 4 illustrates exemplary system features for use with genetic profile testing and comprehensive knowledge-based analysis and reporting in accordance with an embodiment of the present invention; and

FIG. 5 illustrates exemplary system components for use with embodiments of the present invention.

### DETAILED DESCRIPTION OF THE INVENTION

The present invention provides a method and system for generating a comprehensive knowledge-based report, which allows an individual to anonymously order a medical test, such as a genetic profile test, and obtain the results of the test in such a manner that no other unauthorized person can identify or obtain the results.

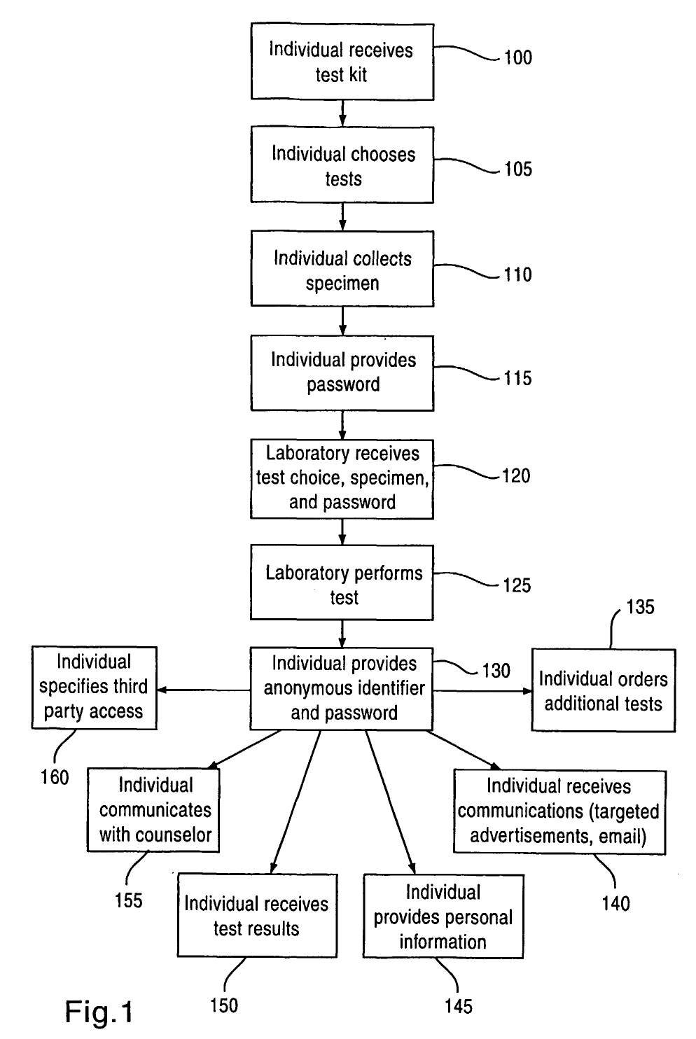

In one embodiment of the present invention, a method and system are provided for the anonymous collection and testing of specimens from persons who desire to keep their identity, and/or the results of the test confidential. FIG. 1 shows that the method can comprise providing a test kit to a patient at a first location, such as a clinic where the test specimen is collected from the patient. At step 100, an individual receives a testing kit, such as a genetic profile testing kit at a clinic or at a physician's office. At the clinic, for example, the name of the patient may be known to the persons who take the patient's personal information, but the method and system according to the invention provides a test kit for each patient, designed such that no one at the clinic will be able to correlate the patient's identity with the test kit provided.

In the embodiment above, the patient at the clinic can select from one or more desired tests, such as a genetic profile test, as shown in step 105 of FIG. 1. Each test kit can contain different types of specimen containers, or receptacles, suitable for use with the specific test desired by the patient. The test kit can also contain instructions on which specimen container to use with which test. The specimen is collected at step 110 of FIG. 1.

The test kit also includes two copies of an anonymous identifier, for example but not limited to, labels imprinted with the bar code or a magnetic strip or combination thereof, a serial number, a user-selected identifier, or Radio Frequency Identifier (RFID) tag, etc, for including with, or on, the patient specimen receptacle for genetic profile testing. This anonymous identifier associates the specimen with the specific patient. Along with the anonymous identifier, a password selection capability, such as a form on which a password is written, is also to be provided in the test kit to permit the patient to select a password at step 115 of FIG. 1. The password is matched with the anonymous identifier so that access to even the test results cannot be obtained without the proper password. The form may also be used if the reporting of the test results is not posted on a secured website, and therefore the form may facilitate the mailing of the test results to the individual.

According to the present invention, the password provides access to the test results, which are identified only by the corresponding identifier that was associated with the patient's specimen. The password selection form and a copy of the bar code remain in the test kit, associated with the specimen. A second copy of the anonymous identifier is retained by the patient. The patient should also memorize or record the password that was selected.

The patient specimen is collected on or in the specimen receptacle and enclosed in the test kit, along with the password selection form and a copy of the anonymous identifier. The anonymous identifier is the only identifying information in the test kit that associates the patient with the specimen and the test results for that specimen. If the results are not to be retrieved by way of a network, such as the Internet, a non-identifiable personal history form may be included with the specimen, which allows answering of questions that are relevant to generating a comprehensive personalized risk assessment report. No other identifying information about the patient is included in the test kit. The test kit is sealed at the clinic, possibly by the patient, for example, after the specimen is collected and enclosed therein, and then sent to a second location, such as a laboratory, where the actual test is performed using the specimen. Step 125 of FIG. 1 shows that the laboratory performs the test.

No one at the laboratory will have access to any patient information, except the anonymous identifier contained in the test kit. In this manner, the identity of the patient cannot be associated with the test results by anyone at the laboratory. At the first location (e.g., sample submission location), although the identity of the patient may be known, neither the anonymous identifier associated with the specimen, nor the patient's password, is known. Thus, anonymity can be assured, since no one at either the first or second location can match the identity of the patient with the patient's specimen, or with the test results from the specimen.

At the laboratory, the selected test, such as a genetic profiling test, is performed on the patient's specimen, and the test results are posted, for example on a secure Internet website that can only be accessed using the patient's identifier and password. This is illustrated at steps 120 and 130 of FIG. 1. Even on the website posting, only the identifier associates any test results with the specimen.

As indicated, both the password and the anonymous identifier are necessary to obtain the test results. In this way, only the patient, or someone to whom the patient has given the password and the anonymous identifier information, can obtain the test results. In other examples of the present invention, posting the test results can be made over the telephone, or using non-network database driven systems. However, whatever the medium by which the test results are provided, one feature of an embodiment of the present invention is that the test results can only be accessed using the patient selected password, and by matching the patient's anonymous identifier to the specific test results.

In the exemplary embodiment described above, the first location (e.g., the clinic) receives the test kits from a second location, such as a laboratory. In another example of the present invention, the clinic and laboratory could be at the same location. For instance, both the clinic and the laboratory can be housed in the same building, or on the same floor of the building, sharing the same office.

In any case, precautions are taken to ensure that the patient's identity cannot be ascertained by persons at the second location, and that neither the anonymous identifier nor the patient selected password can be ascertained by persons at the first location. For example, if housed in the same building, the clinic and laboratory would be separated, and precautions would be taken so that no employees at the clinic, with information about the patient's identity, would be able to share any such information with employees of the laboratory, or vice versa, when the test kit was transferred therebetween so that the anonymity of the patient and the test results will be protected.

Similarly, confidentiality can be maintained during the payment process. For example, payment for services may be made by the patient at the clinic. The clinic would thereafter remit a portion of the payment to the laboratory without providing patient information. Other payment arrangements may also be structured so as similarly to maintain confidentiality.

In another example of the present invention, an individual, such as a patient, can purchase one or more of various tiers of services related to the data and interpretation of data provided to the patient after analysis of the specimen. For example, a patient can purchase two different tiers of medical services related to the data, information and interpretation resulting from a genetic profile test for the specimen. As shown in step 135 of FIG. 1, the individual can order additional tests that constitute such tiers, for example. In particular, in this example, a patient can decide to purchase a service tier that entitles the patient to receive raw results only, or a tier that entitles receipt of raw results, data interpretation and genetic counseling, or a tier that entitles him to raw results, data interpretation, genetic counseling at step 155 of FIG. 1, and secure transmission of his results to a healthcare provider, for example at step 145, wherein the individual provides personal information to a designated person. In the alternative, the individual can designate or specify third-party access of the results, as shown in step 160 of FIG. 1. Of course, such service tiers are not limited to those described here, and one skilled in the art may envisage other variations of these service levels.

In yet another embodiment, the present invention provides a method and system for providing a test kit, such as a genetic profile self test kit. In this embodiment, a test kit is made available or sold to, for example, an individual at a retail outlet, such as a drugstore. The test kit contains a specimen collection medium, such as a buccal swab, and may also contain a specimen storage medium, such as Whatman FTP paper. The test kit also contains a reply card or other password recording medium, which includes a designated password space. The reply card may also include test selection capability that allows the individual to select one or more desired tests from a plurality of available tests. The test kit also contains an anonymous identifier, such as a barcode, identification number, serial number, user-selected identifier, RFID tag, etc. The anonymous identifier may be printed on or included in the reply card, the specimen collection medium, the specimen storage medium, a postage-prepaid envelope, a separate sheet, or in another location that will allow it to be associated with the specimen.

In one exemplary embodiment, the test kit also contains a reminder card for the user to retain. The reminder card includes the anonymous identifier and a reminder password space.

Once the kit is provided, the individual collects one or more specimens, such as by swabbing the inside of the cheek with the buccal swab and blotting the buccal swab onto the Whatman FTA paper. The individual then selects a password and records the password on the reply card and on the reminder card. Next, the individual submits the specimen, together with the reply card. In one embodiment, the individual submits the specimen and the reply card in a postage-prepaid envelope that is included in the test kit. The anonymous identifier may be imprinted directly on the postage-prepaid envelope.

In an alternate example of this embodiment, an individual arrives at a testing location. Medical personnel or other employees collect the sample and provide the individual with the anonymous identifier, and the individual writes the password on the specimen card and seals the specimen envelope.

After the sample is collected, the specimen and/or test kit are received at a laboratory and a test is performed. Remaining specimens or remaining portions of the specimen are stored at the laboratory. If the specimen is received on a buccal swab or includes a vial of blood, for example, the specimen may be refrigerated and stored. If the specimen is received on Whatman FTA paper, a hole may be punched in the paper and a chad thereof may be tested. The remaining paper may be stored. No refrigeration is necessary. An additional advantage of the use of FTA paper is that this type of paper breaks open the cells, achieving the first step in DNA analysis.

Results of the test are then made available to the individual, for example shown in step 150 of FIG. 1. In order to access the results, the user must present the anonymous identifier and password. In one embodiment, the results of the test are sent back to the drugstore or other retail outlet that sold the test kit. The individual presents the anonymous identifier at the retail outlet and receives the results. In another embodiment, the individual receives the results by telephone, facsimile, or another appropriate medium. In yet another embodiment, the individual uses the anonymous identifier and password to access a secure website that presents the results.

In one embodiment, the results presented to the individual are specific to the kind of testing requested by the user, as specified on the reply card. For example, the individual may request a panel to detect a genetic predisposition to Alzheimer's disease. The panel may include a plurality of tests to detect sequence alterations in relevant portions of a plurality of genes. If the individual is found to carry a mutation in a relevant portion of a gene associated with Alzheimer's, the results will alert the individual to the specific mutation, identify the significance of the mutation, and calculate a relative risk factor. For example, the results may inform the user that the particular mutation increases the risk of Alzheimer's by 20%. If, for example, clinical trials have demonstrated that action can be taken to prevent or retard the progression of this possible condition, the results can alert the user to such studies and reference the recommended action. For example, the studies may recommend that the individual increase folic acid intake.

In addition to performing different levels of tests on the individual, as mentioned above, other factors and/or data, such as environmental factors, and personal factors, may be incorporated into the results of the testing, so as to generate a comprehensive report on the individual.

For example, if an individual is a smoker and it is determined that the individual has a particular mutation (e.g., from genetic profile testing), then the present invention can provide a comprehensive report to the individual, based on the testing results and the smoking factor, indicating that the individual has a much higher risk of illnesses or diseases than a non-smoker or a smoker who has a different genetic profile.

To collect such additional information, one example of the present invention provides a set of queries for the individual, in which the set of queries collect data, information and other relevant information such as information that relates to risk factors of the individual. Based on that information, the knowledge-based algorithms of the present invention generate a result, based on the genetic profile, any environmental data, and any other information (e.g., that the individual has provided), to render a more comprehensive report that is geared specifically towards that individual.

In yet another example of the present invention, the comprehensive knowledge-based assessment incorporates standard laboratory chemical analysis of other biological samples, such as blood, urine, etc. and taking into consideration the results of such tests (e.g., that relate to other risk factors, such as cholesterol, blood chemistry tests, certain proteins and enzymes in the blood) to provide a more comprehensive picture of the individual's health condition.

According to the present invention, once an individual's specimen, such as the individual's DNA, is collected in a first location, such as a clinic, and stored at a laboratory, subsequent tests can be further conducted at a later time. For instance, if an individual at a later time (e.g., after the initial testing and analysis) reports that a weakness of the heart and some form of heart disease has developed, or that the individual has high cholesterol, the individual may communicate this information (e.g., via a secured web site hosted by the clinic, the laboratory, or any location where the first test was conducted), and new tests can be performed to identify preventive or corrective measures (e.g., identifying a new diet that is ideally suited for the individual based on having high cholesterol). The present invention can offer such service without requiring the individual to buy another testing kit or the like, and the analysis may be developed or updated without the need for additional testing (e.g., where the new information affects risk determined by previous testing).

One embodiment of the present invention offers the option to submit a request and make payment remotely (e.g., via a website on the Internet). The payment may be made in any form, such as paying by PayPal via the website. The test results can be confidentially posted (e.g., on the website), so as to be accessible by authorization with password and/or identifier. In addition, one embodiment of the present invention offers optional updates of the knowledgebase, such as updates that affect the report of an individual's profile as new discoveries are made in the science relating to the subject matter of the test performed.

In another example, the comprehensive knowledge-based reporting of the present invention provides drug metabolism studies, such as to identify which drug is ideally suited for the individual and at what dosage. For example, not all individuals react the same to each medication (e.g., some may take one type of medication, such as Tylenol®, for headaches, while others respond better if they take aspirin). Therefore, a drug metabolism analysis of the individual included in the comprehensive profile of the individual can provide a more accurate method for prescribing medication.

The knowledgebase may also take under consideration the results of other test, such as, but not limited to, liver enzyme studies, to identify current weaknesses in liver function, in order to better identify drug candidates that are to be prescribed to the individual. The knowledgebase may also analyze these and other genetic components related to detoxification (e.g., how an individual detoxifies certain substances that are contained in household cleaners or gasoline) so that such information can be included in the overall comprehensive knowledge-based report. For example, every time an individual is exposed to potentially toxic substances or fumes through inhalation or exposure to the skin, the risk of degenerative diseases, cancer, neurological diseases or the like can be increased if the individual's genetic profile indicates a weakness in detoxifying certain substances. Conventional liver function studies may indicate stress on the liver due to the increasing toxicity in the body. Hence, having a drug metabolism profile, as well as other studies and tests, as part of the overall comprehensive knowledge-based reporting can facilitate the prescribing of the right medication for the individual. This information can also be used to identify more accurately the risk of potentially developing an illness or disease.

In addition to drug metabolism studies, the present invention, in another embodiment, also includes analyzing factors regarding the individual's food metabolism in the overall comprehensive knowledge-based report.

The evolution of a family tree can also be a predictive factor usable with the present invention. For example, an individual's mother's family can originate in one region of the world, and the father's family can originate in a completely different area. Based on that information, the individual's food metabolism may have evolved over thousands or even hundreds of thousands of years, based, for example, on a certain typical diet, such as grains, meats, fruits, or any combination thereof, for the geographic location of the ancestors. As such, the individual's body may be better geared towards, for instance, processing carbohydrates, because of the grains eaten by his ancestors, and a diet low in carbohydrates could cause weight gain, or nutritional deficiencies because the body is not processing the other foods consumed in an efficient manner, or vice versa.

Thus, the metabolic profile can be a relevant factor in assessing a specific diet, and perhaps additional supplements may be helpful. In a further embodiment, the present invention allows for the knowledgebase to recommend the ideal diet for the individual, based on the information contained in the knowledgebase. Subscribers can periodically update their profile in the knowledgebase with new test results, such as cholesterol levels, weight and other factors, to allow the system to further refine its recommendations. The invention also allows the data that is collected in the knowledgebase to be compiled, to assist in the development of certain functional foods to be offered to subscribers.

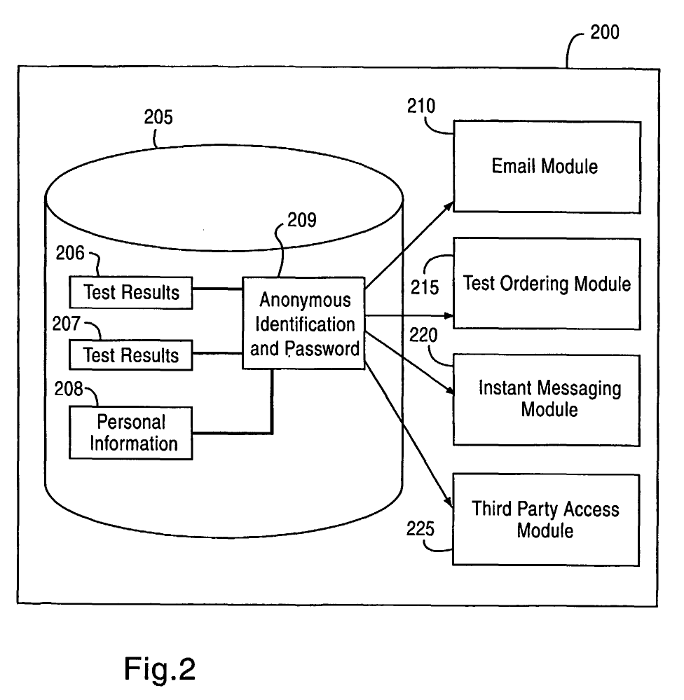

Once the testing and the analysis of all relevant information and factors are gathered/conducted, the present invention, in another embodiment, provides a system, as shown in FIGS. 2, 4 and 5 that allows access to test results and other related data. The present invention may be implemented, in part, using hardware, software or a combination thereof and may be implemented in one or more computer systems or other processing systems. In particular, the preferred embodiment of the invention can operate on an individual's computing system such as a personal computer with supporting applications operating thereon, or the invention can operate on an individual's computing system together with one or more network servers connected to a communication network, such as the Internet, the World Wide Web, Local Area Network, Wide Area Network, wireless communication network, etc., and any combination thereof, with additional supporting applications operating on both the individual's personal computing system and the network server(s) to carry out the functionality described herein.

FIG. 2 illustrates one example of a comprehensive knowledge-based system 200 that includes a data repository 205 that stores the results of genetic and other test that have been conducted on an individual. The comprehensive knowledge-based system 200 of FIG. 2 also includes an email module 210, a test ordering module 215, an instant messaging module 220 and a third party access module 225.

The data repository 205 includes storage units 206, 207, 208 and 209 such as storage memory or database files for storing test results, personal information and anonymous identifiers and passwords of individuals. For example, the test results stored in storage units 206 and 207 are associated with an anonymous identifier and with a password that has been selected by the individual stored in storage unit 209. The test results are not associated with the name of the individual or any other identifying information about the individual.

The email module 210 and the instant messaging module 220 can facilitate communication between all parties involved in comprehensive knowledge-based system, including, but not limited to, clinicians, physicians, individuals/patients, lab technicians, etc.

According to this embodiment, the individual may access the test results by providing the anonymous identifier and the password. The system may also permit the individual to enter non-identifying personal information, such as age, personal and family medical history, personal habits, ethnicity, etc. The non-identifying personal information can be used in conjunction with the results of the test to determine relative risk factors and the likelihood of disease, as well as to inform the individual of the current state of knowledge with respect to countermeasures or possible treatment options that may be discussed with medical professionals.

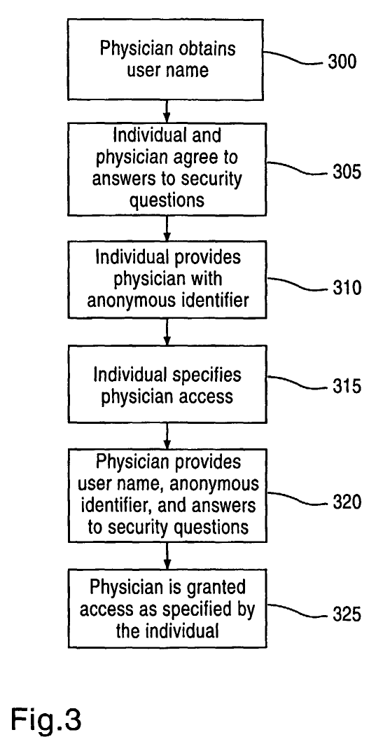

A further embodiment allows the individual optionally to provide full or limited access to a physician or other third party. This feature is provided, for example, by the Third Party Access Module 225 of FIG. 2. Also illustrated, in the flow diagram of FIG. 3, at step 300, a physician obtains an individual's user name. The individual may allow the physician or other third party to view the complete test results, to view partial test results, or to receive answers to selected questions. The physician access assists physicians, for example, in prescribing appropriate drugs or otherwise providing treatment, but it also provides the physician with the ability to input relevant data into the knowledgebase that may assist the system in generating more comprehensive results.

To access information, an individual logs in to the system, for example, by providing the anonymous identification and the password. Once logged in, the individual may see the results of the medical tests. Results include, for example, specific genetic sequences, the significance of the specific genetic sequences, and recommended action, all based on the current state of the knowledgebase.

For instance, the physician, with the individual's consent, has the individual's user name and password. In an alternative embodiment, the physician can answer a set of security questions, as provided in step 305 of FIG. 3, if, for example, the physician does not have the individual's user name and password.

The user name provided with the sample can be, for instance, the bar code number, and the password may be created by the individual. Once this information is provided or determined, the individual can send the DNA specimen and non-identifiable personal information in a self-addressed postage pre-paid envelope, for example. The testing facility receives the envelope and conducts the requisite tests, and the results are posted, for example, on a secure website. The individual logs in with a user name and password, and thereafter can access the report, as provided in steps 320 and 325 of FIG. 3. An additional layer of security may be implemented after the individual logs into the information application of the system. For example, a predetermined set of questions may be presented to the individual or physician, soliciting answers. If the answers match the stored responses, then the individual may be authorized to access the secured information.

The individual can provide the physician with the necessary information, such as the anonymous identifier, as provided in step 310 in FIG. 3. In addition, the physician can have access to an interface specifically for the physician or the physician's nurse, so as to allow access to the genetic information and query the knowledge base. This is shown in step 315 of FIG. 3. The physician can use the physician's own user name and the individual's user name, or an anonymous identifier to log in. The physician can answer a number of different questions that the individual and the physician agree to, for example, and is then authorized to access the records (e.g., once the right combination of answers are provided). Once in the system, the physician can query the system, for example, to determine the drug best suited for the patient (e.g., in the subject matter of hypertension). The present invention can provide the physician with a comprehensive report on hypertension, for example, as it relates to the information provided by the individual, as well as the testing results and other relevant factors. The system can also suggest medication(s) that are ideally suited for the individual, based on the report. In one embodiment, the physician can choose from a list of the recommended medications best suited for the individual, and then be prompted to select the optimum initial dosage to prescribe.

The system also provides functionality that allows the individual to enter non-identifying personal information, such as age, ethnicity, gender, medical history, family history, geographic history, results from other clinical tests and the like. The personal information entered by the individual is stored in the system. The personal information can be used, for example, to analyze, interpret, or refine the results of the medical test. The information can also be used for the system to recommend further specific testing. Many genetic correlations have been proven to be predominant within a particular population segment, such as a population segment that relates to a certain ethnic group. By correlating this type of information the system can recommend testing specific to genetic variations found only in such ethnic groups. Thus, personal information is of importance in providing accurate results.

For example, clinical studies may show that a certain genetic mutation provides an increased risk of osteoporosis only for females. In this case, the personal information provided by the individual can be used to determine the significance of the mutation and the recommended course of action. For example, if an individual with the genetic mutation is female, the significance is that the risk of osteoporosis is increased 40%, and the recommended action is to increase intake of vitamin D. If an individual with the genetic mutation is male, the significance may be none, and the recommended action may be none.

The system also provides functionality to allow an individual to anonymously pose a question to a certified genetic counselor (or other professionals) via email, message board or instant messenger, and for the genetic counselor to respond to the individual without becoming aware of the individual's identity or location. Once an individual has logged into the system, the individual has access to anonymous email and/or instant messaging programs, and the user can send and receive messages from an anonymous email or messaging account that is linked to the anonymous identifier.

Furthermore, the system provides functionality to allow an individual to order additional tests. The Test Ordering Module 215 of FIG. 2 provides this feature. The user may request an additional test and pay anonymously using Paypal or another anonymous payment method. The specimen that is stored in the laboratory may be used for additional tests. The individual accesses the results of the additional tests using the original anonymous identifier and a password, for example.

The specimens stored in the laboratory or the information stored in the system can also be used for research purposes, if authorized by the individual or allowed by law. These purposes can include data mining, population studies, correlation studies, or the like. Studies can be performed by making correlations between existing data in the system or by performing tests on stored samples.

For example, if a new study determines that a particular gene is related to osteoporosis in Iceland, other specimens stored in the laboratory can be tested to determine if the correlation is true in the rest of the population, or in any subsection of the population. In some cases, the system can be queried to find individuals with certain personal information, or with certain genetic predispositions. The system may be queried, for example, to find Asian females with a particular mutation. The specimens belonging to these individuals may then be tested.

As another example, information stored in the system may be examined to find correlations. For example, analysis of the information stored in the system may show that Asian females are 30% more likely to have a particular mutation.

As yet another example, information stored in the system may be used to identify individuals who are good candidates for particular clinical trials or other tests. For example, the makers of a new pain medication may wish to determine whether individuals that are fast metabolizers of aspirin are also fast metabolizers of the new pain medication. In this case, a database in the system can be queried to find individuals who are fast metabolizers of aspirin. Once the individuals who meet a particular set of criteria are identified, they may be contacted to request their participation in the study, for example, via a targeted advertisement or an email within the system. Once an individual has logged in to the system, the user receives emails or targeted advertisements, which are posted, for example, to an anonymous email or messaging account that is linked to the anonymous identifier.

The system also allows an individual to grant selective access to a third party, such as a physician, as provided by module 225 of FIG. 2. The individual may grant access to all test results, grant access to partial test results, or allow the physician to receive answers to specific questions about the results.

For example, physicians treating a patient who has had a drug metabolism or pharmacogenomic profile performed may wish to access the system to determine which drugs are best suited for the patient. However, the patient may not wish to grant the physician access to all test results, and may not even wish to grant the physician access to the pharmacogenomic profile. (For example, any information provided to a physician may become part of the individual's permanent medical record and have an adverse impact on the individual.) In addition, even if the patient grants the physician access to the pharmacogenomic profile, the physician may not have the training to analyze the pharmacogenomic profile and recommend an appropriate drug. Therefore, the invention provides a user-friendly way for physicians to interface with the system to help identify, for example, which drugs to prescribe and what the optimal initial dosage may be.

As a particular example, an individual with hypertension has a pharmacogenomic study performed. The individual would like a physician to prescribe a drug for hypertension that is suited to the individual's genetic profile, without providing the physician complete access to the pharmacogenomic study.

The physician registers with the system and receives a user name. The individual and the physician agree to answers to several security questions, such as the individual's pet's name, favorite color, lucky number, etc. The individual also provides the physician with the anonymous identifier. The individual does not provide the physician with the password.

The individual logs onto the system and specifies the physician's access. The user may specify full access, access to particular test results only, or may allow the physician only to receive answers to specific questions. For example, the individual may specify that the physician may receive only a targeted answer to the question, “Which hypertension drug is best for this individual?” Alternately, the user can specify that the physician enter a diagnosis and query the system to receive a drug recommendation. Other types of third-party access are similarly possible.

As mentioned above, in order to log on to the system, the physician provides the physician's and the patient's anonymous identifiers, along with the answers to the security questions. Depending on the access granted by the individual, the physician can view all results, view a pharmacogenomic profile, view the answer to a targeted question, or enter a diagnosis and receive a recommendation. The recommendation includes, for example, a recommended list of drugs and an initial recommended dosage based on the latest information available.

One example application of the present invention is for prenatal DNA analysis and/or diagnostics, which can include the use of internal control DNA detection of paternally-inherited polymorphisms, such as short tandem repeat (STR) markers, to verify the presence of fetal DNA in the absence of specific mutation detection, thus eliminating false-negative results. The DNA analysis of the present invention may also include proprietary technologies that can be described as Haystack Processing, as well as strategies designed to enrich or purify the fetal DNA, or a portion of it, which will significantly improve the specificity of the test. Haystack Processing relates to methods and compositions that accomplish specific target enrichment in specimens of varying sizes. Haystack Processing is of particular importance where rare targets are sought in a relatively large specimen. For example, fetal RhD status can be detected as early as at eight weeks of gestation using free fetal DNA in maternal plasma from RhD-negative mothers, by assessing the presence of the RhD (+) allele inherited from the father. This analysis would also include detection of STR polymorphisms specific to the Father, to verify the presence of fetal DNA. This approach would allow a definitive result to be obtained, especially in cases where the fetus is RhD negative, because a false-negative result due to the lack of fetal DNA in the specimen would be ruled out by the internal control DNA detection. One additional advantage of the use of STRs as internal controls for the presence of fetal DNA is that it allows for paternity assessment of the fetus, as well. As such, an example of the embodiment can be anonymous paternity testing.

Another example or use of the present invention is for Prenatal DNA diagnostic testing for the assessment of Fragile X syndrome in the fetus. Fragile X syndrome is the most common form of inherited mental retardation and is passed from Mother to fetus. The DNA abnormality is an expansion of a CGG tandem repeat unit within the FMR1 gene located on the X chromosome. This expansion occurs during meiotic processes that form the egg, and thus the mother's DNA will generally not display the expanded STR genotype, while an affected fetus will. Females can carry either a full mutation on one chromosome or a pre-mutation (increased number of repeats but not fully expanded). Pre-mutation alleles have a much greater risk of expanding into a full mutation than do normal alleles. Using free DNA from the maternal serum, the STR repeat number in the FMR1 gene can be assessed, and, if an expanded genotype is found in this sample from a negative (or pre-mutated) mother, this information would indicate that a full mutation is present in the fetus. This test would also include a marker for the human Y chromosome, such as SRY, to assess the sex of the child. This is important because males are affected to a much greater degree than females by this condition, due to the linkage of this gene to the X chromosome. The STR and/or Y chromosome analysis will also serve as the internal control marker to assess the presence of fetal DNA in the sample.

Furthermore, the present method and system can also be used to enrich and/or purify the fetal DNA or relevant portions of it to significantly improve the performance (sensitivity and specificity) of the testing protocols. Differences between the contaminating maternal DNA and the fetal DNA can be used for this purpose. These differences can include, but are not limited to, the DNA sequence itself, the physical form of the DNA (such as size), and differences in the imprinted methylation pattern. The use of Haystack Processing to isolate/concentrate or protect specific DNA sequences has been described extensively. Because the maternally-inherited fetal chromosome will be the same sequence as one of the maternal chromosomes (with the exception of syndromes, such as Fragile X described above), this application may be restricted to sequences inherited from the Father. Even in these cases, however, the performance of the test should be enhanced if these mutations are to be detected in the assay, as in the case of RhD typing, and sensitivity will improve due to the processing of greater amounts of initial specimen, a documented advantage of the Haystack Processing technology.

In one example of the prenatal DNA analysis method and system of the present invention, the fetal DNA may be further enriched or purified using high resolution separation techniques, such as HPLC or capillary acrylamide gel electrophoresis, among others. A complete separation of the fetal DNA from the maternal DNA can allow an unprecedented advantage for prenatal DNA analysis.

An additional difference between the fetal and maternal DNA that can be used for the purpose of purification is the imprinted methylation pattern. It is now known that maternally-inherited DNA differs from paternally-inherited DNA with respect to the pattern of CpG dinucleotides that contain methyl groups. While the presence or absence of a methyl group at the 5-position of the cytosine residue does not change its hybridization properties, and thus makes it indistinguishable from a standard cytosine, treatment of the DNA with the agent sodium bisulfite can convert any unmethylated cytosine to a uracil, which now hybridizes to adenine bases, as opposed to a guanidine. Methylated cytosine residues will be resistant to the conversion, and thus retain the ability to hybridize to guanidine. This reaction specificity can now form the basis for sequence differences resulting from differential methylation patterns that can be used to preferentially isolate the paternally-inherited fetal DNA from the fetal/maternal DNA mixture using a method that can isolate nucleic acids in a sequence-specific manner. One group reported the use of differential DNA methylation as a means to distinguish fetal sequences from maternal DNA in the plasma of the Mother. However, their technique was restricted in its use. Even in the event that only the paternally-derived chromosomes can be isolated by this technique, this approach still represents a significant improvement in the detection methodology, which has relied to date on differential PCR detection of paternally-inherited mutations in a large background of maternal DNA.

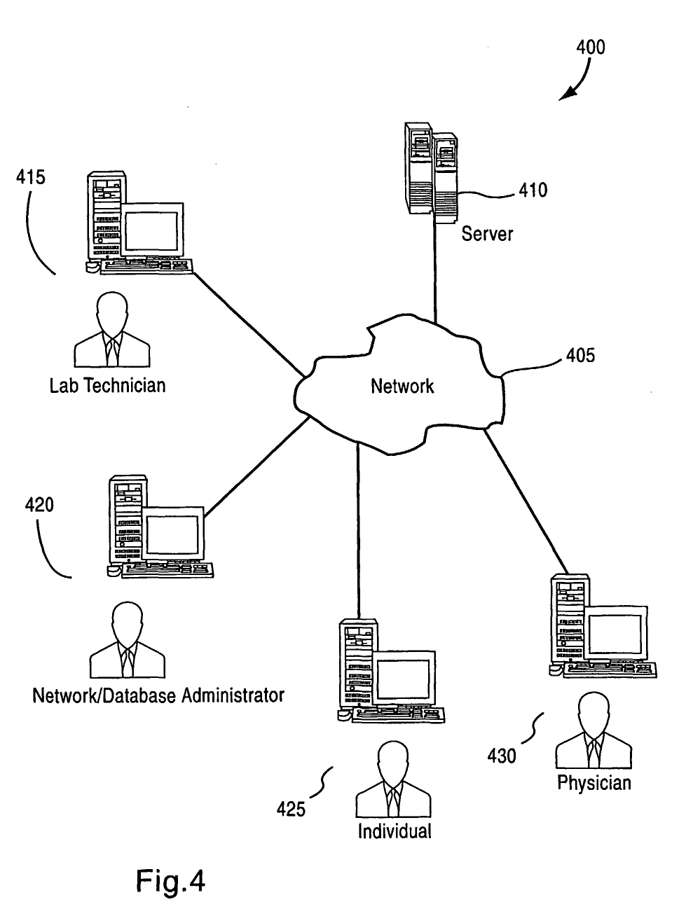

FIG. 4 shows a comprehensive knowledge-based network system 400 including a communication network 405, one or more network servers 410, and various locations, sites, computing stations and/or nodes 415, 420, 425, 430 connected to the network. For example, FIG. 4 illustrates the various workstation of the communication network 405 as being a laboratory technician station 415, a network/database administrator station 420, an individual station 425 and a physician's office 430. The communication network 405 can include the Internet, the World Wide Web network, wireless communication network, Local Area Network, Wide Area Network, or any communication network to facilitate electrical, digital and analog communication signals between the nodes of the network.

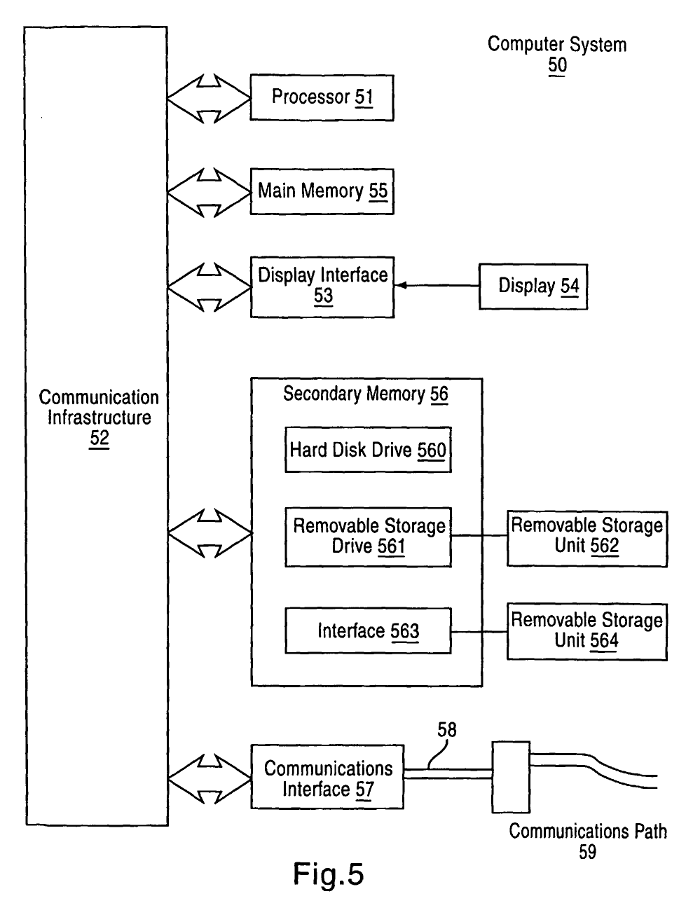

FIG. 5 illustrates a computer network system 50 that includes one or more processors, such as processor 51 for use in operation of various embodiments of the present invention. The processor 51 is connected to a communication infrastructure 52 (e.g., a communications bus, cross-over bar, or communication network). Various software embodiments are described in terms of this exemplary computer system. After reading this description, it will become apparent to a person skilled in the relevant art(s) how to implement the invention using other computer systems and/or architectures.

Computer system 50 can include a display interface 53 that forwards graphics, text, and other data from the communication infrastructure 52 (or from a frame buffer not shown) for display on the display unit 54. Computer system 50 also includes a main memory 55, preferably random access memory (RAM), and may also include a secondary memory 56. The secondary memory 56 may include, for example, a hard disk drive 560 and/or a removable storage drive 561, representing a floppy disk drive, a magnetic tape drive, an optical disk drive, or the like. The removable storage drive 561 reads from and/or writes to a removable storage unit 562. Removable storage unit 562, represents a floppy disk, magnetic tape, optical disk, or the like, which is read by and written to removable storage drive 561. As will be appreciated, the removable storage unit 562 includes a computer usable storage medium having stored therein computer software and/or data.

In alternative embodiments, secondary memory 56 may include other similar devices for allowing computer programs or other instructions to be loaded into computer system 50. Such devices may include, for example, a removable storage unit 564 and an interface 563. Examples of such may include a program cartridge and cartridge interface (such as that found in video game devices), a removable memory chip (such as an erasable programmable read only memory (EPROM), or programmable read only memory (PROM)) and associated socket, and other removable storage units 564 and interfaces 563, which allow software and data to be transferred from the removable storage unit 564 to computer system 50.

Computer system 500 may also include a communications interface 57. Communications interface 57 allows software and data to be transferred between computer system 50 and external devices such as servers, other computer systems, mobile device, etc. Examples of communications interface 57 may include a modem, a network interface (such as an Ethernet card), a communications port, a Personal Computer Memory Card International Association (PCMCIA) slot and card, etc. Software and data transferred via communications interface 57 are in the form of signals 58, which may be electronic, electromagnetic, optical or other signals capable of being received by communications interface 57. These signals 58 are provided to communications interface 57 via a communications path (e.g., channel) 59. This path 59 carries signals 58 and may be implemented using wire or cable, fiber optics, a telephone line, a cellular link, a radio frequency (RF) link and/or other communications channels. In this document, the terms “computer program medium” and “computer usable medium” are used to refer generally to media such as a removable storage drive 561, a hard disk installed in hard disk drive 560, and signals 58. These computer program products provide software to the computer system 50. The invention is directed to such computer program products.

Computer programs (also referred to as computer control logic) are stored in main memory 55 and/or secondary memory 56. Computer programs may also be received via communications interface 57. Such computer programs, when executed, enable the computer system 50 to perform the features of the present invention, as discussed herein. In particular, the computer programs, when executed, enable the processor 51 to perform the features of the present invention. Accordingly, such computer programs represent controllers of the computer system 50.

While the invention has been described in detail in particular embodiments using specific examples, it would be appreciated by those skilled in the art that various modifications of those details could be developed in light of the overall teaching of the disclosure. For example, while the invention has been described in terms of a method and system for genetic testing, the invention is equally applicable to other types of testing. Similarly, while the invention has been described in terms of a method and system for confidential testing, alternate embodiments of the invention may not preserve confidentiality. Embodiments of the invention may include, for example, non-confidential methods and systems for allowing an individual to order tests and interpret test results. Therefore, the particular embodiments disclosed herein are intended to be illustrative only and not limiting to the scope of the invention.

1. A method of genetic profile testing, comprising:
collecting a specimen for testing;
associating an identifier with the specimen;
performing a genetic profile test on the specimen;
analyzing data resulting from the genetic profile test; and
providing an analysis report of the genetic profile test in response to receipt of the identifier.

2. The method of claim 1, further comprising:
providing a genetic profile testing kit at a first location.

3. The method of claim 2, wherein the first location comprises a clinic, a drug store, or a physician's office.

4. The method of claim 1, further comprising:
associating a password with the specimen;
providing the analysis report in response to receipt of the identifier and the password.

5. The method of claim 4, further comprising:
providing the analysis report via a communication network comprising a telephone network, the Internet, and a database driven Local Area Network.

6. The method of claim 5, further comprising:
providing access to the analysis report via the communication network to an accessor selected from a group consisting of a physician, a provider of the specimen, a clinician, and a laboratory technician.

7. The method of claim 1, further comprising:
issuing an invoice for the analysis report.

8. The method of claim 1, wherein the identifier is selected from a group consisting of a bar code, a magnetic strip, a serial number, and a Radio Frequency Identifier (RFID) tag.

9. The method of claim 1, wherein performing a genetic profile test on the specimen comprises performing prenatal DNA analysis or diagnostics.

10. The method of claim 9, further comprising:
determining fetal DNA in maternal plasma or urine.

11. The method of claim 1, wherein the identifier is an anonymous identifier.

12. The method of claim 1, wherein the identifier is received from an accessor selected from a group consisting of a provider of the specimen, a physician, a clinician, and a laboratory technician.

13. A comprehensive knowledge-based reporting method, comprising:
receiving a specimen for testing;
associating an identifier with the specimen;
performing a genetic profile test on the specimen;
analyzing data resulting from the genetic profile test;
compiling a plurality of relevant factors associated with the specimen;
providing a comprehensive analysis report based on the genetic profile test and the plurality of relevant factors to an individual having the identifier.

14. The method of claim 13, wherein compiling the plurality of relevant factors includes compiling at least one of environmental, personal, family, medical, and behavioral history of a provider of the specimen.

15. The method of claim 13, wherein compiling the plurality of relevant factors includes compiling at least one of drug metabolism studies, food metabolism studies, and chemical analysis of a provider of the specimen.

16. The method of claim 13, further comprising:
associating a password with the specimen; and
providing the comprehensive analysis report in response to receipt of the identifier and the password.

17. The method of claim 16, further comprising:
providing the comprehensive analysis report via a communication network comprising a telephone network, the Internet, and a database driven Local Area Network.

18. The method of claim 17, further comprising:
providing access to the comprehensive analysis report via the communication network to an accessor selected from a group consisting of a physician, a provider of the specimen, a clinician, and a laboratory technician.

19. The method of claim 13, further comprising:
issuing an invoice for the comprehensive analysis report.

20. The method of claim 13, wherein the identifier is selected from a group consisting of a bar code, a magnetic strip, a serial number, and a Radio Frequency Identifier (RFID) tag.

21. The method of claim 13, wherein performing a genetic profile test on the specimen comprises performing prenatal DNA analysis or diagnostics.

22. The method of claim 21, further comprising:
determining fetal DNA in maternal plasma or urine.

23. The method of claim 13, wherein the identifier is an anonymous identifier.

24. A confidential testing and reporting system, comprising:
a test kit, the test kit including:
a specimen collector; and
an identifier submission mechanism;
a specimen receiving facility; and
a confidential testing and reporting mechanism, the confidential testing and reporting mechanism including:
a specimen analyzer to produce a specimen test result;
an accessing mechanism for confidentially accessing the specimen test result; and
a providing mechanism for providing at least one level of optional information relating to the test result.

25. The system of claim 24, wherein the specimen collector includes a specimen collection medium.

26. The system of claim 24, wherein the specimen collector includes a specimen storage medium.

27. The system of claim 24, wherein the identifier submission mechanism comprises a reply card.

28. The system of claim 24, wherein at least one level of optional information includes at least one tier of health related information relating to the test result.

29. The system of claim 24, wherein the confidential testing relates to genetic testing.

30. The system of claim 24, wherein the identifier submission mechanism includes a mechanism for selecting at least one type of test to be performed.

31. The system of claim 24, wherein the test kit includes an anonymous identifier.

32. The system of claim 31, wherein the anonymous identifier is selected from a group consisting of a bar code, an identification number, a serial number, a radio frequency identification tag, and a user-selected identifier.

33. A confidential information collection and reporting system, comprising:
a first mechanism for confidentially collecting genetic or diagnostic test results;
a storage medium for storing the genetic or diagnostic test results;
a second mechanism for querying the database to ascertain a disposition of a predetermined individual related to a medical condition; and
a third mechanism for providing a report of the database query to the predetermined individual or to a third party associated with the predetermined individual.

34. The system of claim 33, wherein the report of the database query comprises a proposed behavioral modification to treat or prevent the medical condition.
